(19) United States
(12) Patent Application Publication
(10) Pub. No.: US 2007/0004796 A1
(43) Pub. Date: Jan. 4, 2007

(54) Processes for the preparation of protected-(+)-catechin and (-)-epicatechin monomers, for coupling the protected monomers with an activated, protected epicatechin monomer, and for the preparation of epicatechin-(4B,8)-epicatechin or -catechin dimers and their digallates

(76) Inventors: Leo Romanczyk JR., Hackettstown, NJ (US); Pradeep K. Sharma, Westford, MA (US); Alexander G. Kolchinski, Winchester, MA (US); Helene A. Shea, Rutland, MA (US); Yanni Gou, Acton, MA (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 11/169,860

(22) Filed: Jun. 29, 2005

(51) Int. Cl. A61K 31/353; C07D 311/02

(52) U.S. Cl. 514/456; 549/405

(57) ABSTRACT

Improved processes for the preparation of tetra-O-benzyl protected catechin, for the coupling of the tetra-O-benzyl protected catechin or epicatechin with a C-4 activated, tetra-O-benzyl protected epicatechin for the galloylation of the epicatechin-(4β,8)-catechin or -epicatechin dimer-the dimer digallates, and for the deprotection (i.e., debenzylation) of the protected epicatechin dimers and protected epicatechin dimer digallates are disclosed.

### BACKGROUND OF THE INVENTION

1. Field of the Invention

This invention relates to improved processes for the preparation of protected catechin and epicatechin monomers, for their coupling with a C-4 activated, protected epicatechin monomer to form protected procyanidin (4β,8)-dimers, and for the preparation of the procyanidin (4β,8) dimer digallates, and for the preparation of the procyanidin (4β,8) dimers.

2. Discussion Of Related Art

The preparation of 5,7,3′,4′-tetra-O-benzyl-(+)-catechin is exemplified in U.S. Pat. No. 6,864,377 issued Mar. 8, 2005 to L. J. Romanczyk, Jr. et al. (+)-Catechin dissolved in DMA was protected by benzylation with benzyl bromide in the presence of potassium carbonate.

The preparation of (2R)-5,7,3′,4′-tetrakis (benzyloxy) flavan-3-one is exemplified in the U.S. Pat. No. 6,420,572 issued Jul. 16, 2002 to Leo J. Romanczyk et al., as well as U.S. Pat. No. 6,528,664 issued Mar.4, 2003 to L. J. Romanczyk, Jr. et al., and U.S. Pat. No. 6,849,746 issued Feb. 1, 2005 to Leo J. Romanczyk, et al. See Example 2 where 5,7,3′,4′-tetra-O-benzyl-catechin in methylene is oxidized at room temperature using freshly prepared Dess-Martin periodinane (DMP) reagent.

The preparation of 5,7,3′,4′-tetra-O-benzyl-epicatechin by the reduction of (2R)-5,7,3′,4′-tetrakis(benzyloxy)flavan-3-one is exemplified in the '572, '664, and '746 patents. See Example 3 where the reduction is carried out with lithium tri-sec-butylborohydride and lithium bromide in anhydrous tetrahydrofuran.

The preparation of 4-(2-hydroxyethoxy)-5,7,3′,4′-tetra-O-benzyl-(−)-epicatechin is exemplified in the '572, '664, and '746 patents. See Example 4 where 5,7,3′,4′-tetra-O-benzyl-epicatechin is reacted with ethylene glycol in anhydrous methylene chloride in the presence of 2,3-dichloro-5,6-dicyano-1,4-benzoquinone and 4-dimethylaminopyridine. See also Example 1 of U.S. 2004/0116718 published Jun. 17, 2004 and U.S. 2005/0020512 A1 published Jan. 27, 2005 naming A. P. Kozikowski et al. as inventors.

See Example 5 where 4-(2-hyrdoxyethoxy)-5,7,3′,4′-tetra-O-benzyl-epicatechin was coupled with 5,7,3′,4′-tetra-O-benzyl-epicatechin in anhydrous tetrahydrofuran and anhydrous methylene chloride using titanium tetrachloride in methylene chloride as the Lewis acid. See also Example 2, Part B of the '512 published U.S. patent application where 4-(2-hydroxyethoxy)-5,7,3′,4′-tetra-O-benzyl-epicatechin was coupled with 5,7,3′,4′-tetra-O-benzyl-epicatechin in anhydrous methylene chloride using Bentonite K10 clay as the Lewis acid.

Preparation of, 5,7,3′,4′-tetra-O-benzyl-epicatechin-(4β,8)-5,7,3′,4′-tetra-O-benzyl-epicatechin is exemplified in the '842, '572, '664 and '746 patents. See Example 5 where tetra-O-benzyl-4-(2-hydroxyethoxy)-epicatechin is coupled with tetra-O-benzyl-epicatechin in an anhydrous mixture of tetrahydrofuran and methylene chloride in the presence of titanium tetrachloride. The oligomers are isolated by column chromatography on silica gel. Elution is carried out with a mixture of dichloromethane:hexane:ethyl acetate (13:13:1). The dimer and trimer are further purified by preparative HPLC on a silica gel column using ethyl acetate:hexane or ethyl acetate:isooctane as the eluant. See also Example 2, Part B of the published '512 U.S. application where the coupling is carried out in anhydrous methylene chloride in the presence of Bentonite K-10 clay as the Lewis acid.

Removal of the benzyl protecting groups from 5,7,3′,4′-tetra-O-benzyl-epicatechin-(4β,8)-5,7,3′,4′-tetra-O-benzyl-epicatechin is disclosed in the '572, '664 and '746 patents. See Example 6 where the 5,7,3′,4′-tetra-O-benzyl-epicatechin-(4β,8)-5,7,3′,4′-tetra-O-benzyl-epicatechin dimer in an ethyl acetate-methanol mixture was debenzylated using 10% palladium on carbon and 1 bar of hydrogen. The crude dimer is purified by preparative HPLC.

Galloylation of 5,7,3′,4′-tetra-O-benzyl-epicatechin-(4β,8)-5,7,3′,4′-tetra-O-benzyl-epicatechin is exemplified in the '842, '572, '664 and '746 patents. See Example 7 where tri-O-benzyl-gallic acid is reacted with 5,7,3′,4′-tetra-O-benzyl-(4p,8)-5,7,3′,4′-tetra-O-benzyl dimer.

Debenzoylation of the protected epicatechin (4β,8) dimer digallate is exemplified in the '842, '572, '664 and '746 patents discussed above, See Example 8 where the protected dimer digallate was debenzoylatd in a mixture of tetrahydrofuran, methanol, and water using 20% palladium hydroxide on carbon and 1 bar of hydrogen. The crude product is purified by preparative HPLC.

There is a need for developing improved processes capable of manufacturing large quantities of tetra-O-benzyl-(+)-catechin, tetra-O-benzyl-(−)-epicatechin, epicatechin-(4β,8)-catechin, and 3-O-galloyl-epicatechin-4β,8-(3-O-galloyl-epicatechin), particularly processes which produce compounds having an HPLC purity of at least 95% or greater and processes which can use typical equipment found in a manufacturing plant.

### SUMMARY OF THE INVENTION

Benzylation

An improved process for the room temperature benzylation of 5,7,3′,4′-tetra-O-benzyl-(+)-catechin involves the use of 5.0 equivalents of benzyl bromide. The benzylation is carried out in the presence of potassium carbonate (7.5 equivalents) using dimethylformamide (1 g/10 ml) as the solvent. The benzyl bromide is added over 6 hours keeping the internal temperature less than 30° C. and stirred for about 18 to about 24 hours at room temperature. The crude product is purified by dissolution in hot trichloroethylene. The solution is allowed to cool to room temperature and then cooled from −20° C. to −26° C. for about 56 hours. The solids are suction filtered, washed with cold trichloroethylene and heptane and vacuum dried. The yield is about 46%. The HPLC purity is about 97.76%.

Oxidation

The oxidation is only successful under Dess-Martin periodinane conditions. The crude reaction mixture is incubated to remove the Dess-Martin reduction by-product using a mixture of hot methylene chloride and methanol without purification by silica gel chromatography.

Stereoselective Reduction

An improved process for the preparation of 5,7,3′,4′-tetra-O-benzyl-(−)-epicatechin involves the stereoselective reduction of (2R)-5,7,3′,4′-tetrakis-(benzyloxy)-flavan-3-one using cesium carbonate as the base in conjunction with ruthenium-(R)-(+)-2,2′-bis(diphenylphosphino)-1,1′-binaphthyl. Preferably, 10 mol % of cesium carbonate and 10 mol % of ruthenium-(R)-(+)-2,2′-bis(diphenylphosphino)-1,1′-binaphthyl are used. The reduction is carried out in tetrahydrofuran at about 40° C. to about 75° C. and about 200 psi hydrogen for about 16 to about 63.5 hours, with the longer time being used at the lower temperature. The yield is calculated to be 82% based on HPLC.

Another improved process for the stereoselective reduction of (2R)-5,7,3′,4′-tetrakis-(benzyloxy)-flavan-3-one involves the use of aluminum isopropoxide in toluene and 2-propanol under Meerwein-Ponndorf-Verley conditions. The crude product is purified by trituration with methanol at room temperature which increases the diastereometric selectivity from about 26:1 to about 92:1. Alternatively, the crude product is crystallized directly from the concentrated reaction mixture and then triturated with methanol at 50° C. which also increases the diastereometric selectivity to about 650:1. The yield is about 80%. The purity is about 98%.

Coupling

An improved process for preparing a 5,7,3′,4′-tetra-O-benzyl-epicatechin-(4β,8)-(5,7,3′,4′-tetra-O-benzyl-catechin) dimer or a 5,7,3′,4′-tetra-O-benzyl-epicatechin-(4β,8)-5,7,3′,4′-tetra-O-benzyl-epicatechin) dimer involves coupling at least one equivalent of 4-(2-hydroxyethoxy)-5,7,3′,4′-tetra-O-benzyl-epicatechin with four equivalents of 5,7,3′,4′-tetra-O-benzyl-(+)-catechin or with four equivalents of 5,7,3′,4′-tetra-O-benzyl-(−)-epicatechin at 0° C. using Bentonite K-10 clay as a Lewis acid catalyst. The coupling is carried out in dichloromethane under a nitrogen atmosphere. The majority of the monomer (70-80%) is crystallized out using ethyl acetate, thus easing the separation of the monomer, dimer, and trimer via silica gel chromatography. The benzylated (4β,8) dimer is isolated after silica gel chromatography at 100-150 psi pressure using a mixture of heptane and ethyl acetate as an eluant followed by preparative HPLC. The yield is about 72% to about 76% for the 5,7,3′,4′-tetra-O-benzyl-epicatechin-(4β,8)-5,7,3′,4′-tetra-O-benzyl-catechin dimer and about 72 to about 80% for the 5,7,3′,4′-tetra-O-benzyl-epicatechin-(4β,8)-5,7,3′,4′,-tetra-O-benzyl-epicatechin dimer. The HPLC purity of the tetra-O-protected-(4β,8)-tetra-O-protected catechin dimer is greater than about 96%. The HPLC purity of the tetra-O-protected epicatechin-(4β,8)-tetra-O-protected epicatechin dimer is greater than 97%.

Debenzylation

An improved process for deprotecting, i.e., debenzylating, the dimer involves the room temperature hydrogenation of 5,7,3′,4′-tetra-O-benzyl-catechin-(4β,8)-5,7,3′,4′-tetra-O-benzyl-epicatechin or 5,7,3′4′-tetra-O-benzyl-epicatechin-(4β,8)-5,7,3′,4′-tetra-O-benzyl-epicatechin using of a biphasic solvent system consisting essentially of ethyl acetate, methanol, and water or preferably of ethyl acetate and water (1:30 v/v). The hydrogen pressure is about 15 psi. Excess palladium hydroxide on carbon is used as the catalyst. The preferred amount is 30 wt. % loading of palladium hydroxide on carbon (20 wt. %, 50% wet). The dimer is isolated by reverse phase preparative HPLC. The aqueous layer from the reaction mixture is washed with an organic solvent and lypholized. The dimer yield is near quantitative. The HPLC purity is greater than about 95%.

Recovery of Dimer Digallate

An improved process for the recovery of epicatechin-(4β,8)-epicatechin dimer digallate or epicatechin-(4β,8)-catechin dimer digallate [same question as above] after the room temperature hydrogenation of 5,7,3′,4′-tetra-O-benzyl-3-O-(3,4,5-tri-O-benzylgalloyl)-epicatechin-(4β,8)-[5,7,3′,4′-tetra-O-benzyl-3-O (3,4,5-tri-O-benzylgalloyl)-epicatechin] in the presence palladium hydroxide on carbon involves filtering the reaction mixture and washing the filter cartridge with water, ethyl acetate, and again with water. The washings are combined in a separatory funnel and hexane is added. Preferably, the cartridge is rewashed with ethyl acetate and warm water (25°-30° C. for dimer digallates). The aqueous and organic layers are separated. The organic layer is washed with water. The aqueous layer is separated and combined with the other aqueous layers and lyophilized for about 72 hours. The yield is about 80%. The HPLC purity is about 98%.

### DESCRIPTION OF PREFERRED EMBODIMENTS

A. Purification of 5,7,3′,4′-Tetra-O-Benzyl-(−)-Catechin

Trichloroethylene is the best solvent for purifying the crude material. When the crude product is dissolved in hot trichloroethylene (1 g/10 ml), and cooled to −20° C. for about 18 hours a white solid results. The yield is 46%. The HPLC purity is 96.81%. There is one major impurity (2.33%) and three minor impurities (<0.77%). Using only 5 volumes of hot trichloroethylene (1 g/5 mL) increases the yield to 60% without adversely affecting the purity (96.42%). Cooling the solution to room temperature does not produce any precipitate.

Other solvents are not suitable either because the yield is poor and/or the purity is poor. Information on the solvents, yields, purities, and number of impurities is set out below.

| | | HPLC | |
|---|---|---|---|
| | | Purity | |
| Solvent | Yield | (% AUC) | Impurities |
| hot ethanol (1 g/20 mL) | 88% | 63.8 | mixture 36.2 |
| hot ethyl acetate: ethanol 20%:80% | 62% | 67.01 | 1 major |
| v/v) | | | (31.16%) |
| methylene chloride (1 g/3 ml) at room | 40% | 89.75% | 1 major |
| temperature, followed by hot tert- | | | (7.67%) |
| butylmethyl ether (30 mL) | | | 4 minor |
| hot tert-butylmethyl ether (1 g/10 mL) | 42% | 84.05% | 4 minor |
| TBME | | | |
| hot toluene (1 g/5 mL) | 50% | 76.19% | 1 major |
| | | | (20.75%) |
| | | | 6 minor |
| hot benzotrifluoride (1 g/15 mL) | 90% | 73.01% | 1 major |
| | | | (26.37%) |
| | | | 6 minor |

B. Oxidation of 5,7,3′,4′-Tetra-O-Benzyl-(+)-Catechin

The oxidation of the C-3 hydroxy group of 5,7,3′,4′-tetra-O-benzyl-(+)-catechin is necessary in order to perform the stereoselective reduction of the 3-ketone, i.e., the (2R)-5,7,3′,4′-tetrakis(benzyloxy)flavan-3-one, to 5,7,3′,4′-tetra-O-benzyl-(−)epicatechin. The oxidation conditions are very specific for this molecule and the oxidation is only successful under Dess-Martin periodinane conditions. The crude reaction mixture is incubated to remove the Dess-Martin reduction by-product using a mixture of hot methylene chloride and methanol. Previously the crude product was purified by silica gel chromatography which can now be eliminated.

The following oxidative methods do not work.

| Method of Oxidation | Results |
|---|---|
| Standard Swern Oxidation | No product as judged by |
| | HPLC and TLC analysis |
| Modified sodium hypochlorite and | Starting material consumed |
| 2,2,6,6-tetramethyl-1-piperidinyloxy, | number of spots on TLC plate |
| free radical conditions at pH > 8 | |
| Stabilized 2-iodobenziodooxale oxide | Number of side products |
| in methylene chloride at RT or reflux | |
| PtO₂ | Unsuccessful |
| N-methyl morpholine and tetrapropyl | Unsuccessful |
| ammonium peruthenate | |
| Pt/Bi | Unsuccessful |
| (MnO₂) | Unsuccessful |

C. Stereoselective Reduction of (2R)-5,7,3′,4′-Tetrakis(benzyloxy)flavan-3-one

The stereospecific reduction of 5,7,3′,4′-tetra-O-benzyl-(+)-catechin is successful under Meerwein-Ponndorf-Vorley reduction conditions using aluminum isopropoxide as a catalyst and isopropyl alcohol as a reducing agent in toluene as the solvent. See Wilds, A. L., Org. React. 1944, 2, 17. Following the conditions taught in the Wilds et al. article results in a mixture of Bn₄EC and Bn₄C in a ratio of 89:7. When the conditions are modified by distilling the acetone formed during the reduction to force the reaction to completion, the desired product is isolated in 88% yield having 98.7% HPLC purity (only 1% 5,7,3′,4′-tetra-O-benzyl-(+)-catechin) after crystallization/trituration of the crude product with toluene and methanol.

The desired product is also obtained in good yield when 10 mole % of ruthenium(II)-(R)-(+)-2,2′-bis(diphenylphosphino)-1,1′-binaphthyl [Ru(II)-R-BINAP] is used as a catalyst in the presence of 10 mole % cesium carbonate (Cs CO₃)at 50° C. and 75° C. under 200 psi of hydrogen for ˜63.5 h in tetrahydrofuran. The yield is about 82% of the protected (−)-epicatechin. Only about 5% of the falvan-3-one starting material is recovered. Catalysts which are not useful for the reduction of the C-3 ketone are set out below.

| Catalyst* | Solvent | H₂/° C. | Product |
|---|---|---|---|
| [dppb) Ru(II)Cl₂ | THF | 50 psi | 0% |
| [II-Mol-BINAP] | THF | 50 psi 50° C. | 5% |
| Cs₂ CO₃ and | THF | 50 psi 50° C. | 0% |
| [dppb)Ru(II)Cl₂ | | 7 h | |
| 5% Ru/C or 5% Rh/C | THF/MeOH | 100 psi 50° C. | 0% |
| | 1/1, v/v | 63.5 h | |
| 5% Pt/C | THF/MeOH | 100 psi 50° C. | 0% |
| | 1/1, v/v | 63.5 h | |
| Cs₂CO₃ and [(II)-R-BINAP] | THF | 200 psi 50° C. | 82% |
| | | 63.5 h | |
| [Ru(II)-(Ts DPEN)] and | THF/IPA | hydrogen | 19% |
| KOH | | transfer | |
| | | 23-80° C. 15 h | |
| [Ru(II)-R-BINAP] and | THF | 50 psi 50° C. | 1% |
| Cs₂CO₃ | | 16 h | |
| [Ru(II)-R-BINAP] and | THF | 200 psi 50° C. | 9% |
| Cs₂CO₃ | | 16 h | |
| [Ru(II)-(dppb] and | THF | 50 psi 50° C. | 0% |
| Cs₂CO₃ | | 16 h | |
| Ru(II)-(dppb] and Cs₂CO₃ | THF | 200 psi 50° C. | 0.3% |
| | | 16 h | |
| Ru(II)-R-BINAP] and | THF | 50 psi 75° C. | 16% |
| Cs₂CO₃ | | 4 h | |
| Ru(II)-R-BINAP] and | THF | 200 psi 75° C. | 25% |
| Cs₂ CO₃ | | 4 h | |
| Ru(II)-(dppb] | THF | 50 psi 50° C. | 0.75% |
| | | 4 h | |
| [Ru(II)-(dppb] | THF | 200 psi 75° C. | 1.4% |
| | | 4 h | |
| [Ru(II)-R-BINAP] | THF | 200 psi 75° C. | 42% |
| [(2R)-(-)-1,1,-bis- | | 16 h | |
| (4-methoxypheyl)-3-methyl- | | | |
| 1,2-butanediamine] | | | |
| and Cs₂CO₃ | | | |
| RuCl₂(PPH₃)₂ with and | THF | 200 psi 75° C. | 8% |
| without Cs₂CO₃ | | 16 h | and |
| | | | 19% |
| RuCl₂(PPh₃)₂ | IPA | 200 psi 80° C. | 9% |
| | | 16 h | |
| RuCl₂(PPh₃)₂ and Li Br | IPA/THF | 200 psi 85° C. | 2% |
| | | 16 h | |
| RuCl₂(PPh₃)₂ and KOH | IPA/THF | 200 psi 80° C. | 9% |
| | | 16 h | |
| Rh (1,1′-bis(diphenyl- | THF/MeOH | 200 psi 75° C. | 8% |
| phosphinoferrocene) | | 16 h | |
| and Cs₂CO₃ | | | |
| Ru₂Cl₂(PPh₃)₂ and Cs₂CO₃ | THF/IPA | hydrogen | very |
| | | transfer | little |
| | | 85° C. 16 h | |
| RuCl₂(PPh₃)₂ and KOH | IPA/THF | hydrogen | very |
| | | transfer | little |
| | | 85° C. 16 h | |
| Rh (COD)(1,1′-bis | THF/MeOH | 200 psi 75° C. | 10% |
| (diisopropyl- | | 16 h | and |
| phosphinoferrocene)BF₄ | | | 21% |
| with and without CS₂CO₃ | | | |
| [Ru(II)-R and S-BINAP] | THF | 200 psi 75° C. | 27% |
| and Cs₂CO₃ | | 16 h | |
| [Ru(II)-(TsDPEN)] | THF/IPA/ | hydrogen | 0% |
| and KOH | acetone | transfer | |
| | | 80° C. 15 h | |

*dppb is an abbreviation for 1,4-bis(diphenylphosphino)butane and BINAP is an abbreviation for 2,2′-bis(diphenylphosphino)-1,1′-binapthyl.

D. Purification of 4-(2-Hydroxyethoxy)-5.7,3′,4′-Tetra-O-Benzyl-(−)-Epicatechin

The 4-(2-hydroxyethoxy)-5,7,3′,4′-tetra-O-benzyl-epicatechin is prepared by reacting 5,7,3′,4′-tetra-O-benzyl-epicatechin with ethylene glycol in methylene chloride using excess 4-dimethylaminopyridine and 2,3-dichloro-5,6-dicyano-1,4-benzoquinone. Carrying out the reaction for 18 h at 65° C. with 2-iodobenziodooxole oxide using ethylene glycol in dimethylsulfaxide (DMSO) rather than methylene chloride does not produce the desired product. The use of 4-dimethylaminopyrine and ethylene glycol in methylene chloride also does not produce the desired product. Even though the synthesis cannot be improved, the purification of the desired compound can be.

First, the desired compound is purified by single silica gel column chromatograph. The silica gel slurry is prepared by adding silica gel to the reaction mixture and drying under vacuum. The mixture is placed on top of a silica gel column. The product is eluated with heptane:ethyl acetate (2:1, v/v). Fractions containing pure product, as judged by TLC and HPLC analyses, are combined and the solvent is removed under vacuum. The purified product is then dissolved in boiling ethyl acetate, cooled, and diluted with heptane. The resulting suspension is vigorously stirred overnight at RT. The solid is filtered, heptane washed, and dried in vacuum.

E. Coupling The Benzyl-Protected Monomers With the Activated, Benzyl-Protected Epicatechin or Catechin Monomer

Large excesses of the 5,7,3′,4′-tetra-O-benzyl-epicatechin or the 5,7,3′,4′-tetra-O-benzyl-catechin monomers are used. The dimer is separated from the reaction mixture containing the unreacted 5,7,3′,4′-tetra-O-benzyl-epicatechin monomer or 5,7,3′,4′-tetra-O-benzyl-catechin monomer and the resulting benzylated (4β,8) trimer using a single silica gel column at a 100-150 psi, 19.60 g silica gel on a 30-cm column, a flow rate of 600-650 mL/min of ethyl acetate: heptane (1:3, v/v), monitoring at 280 nm, and a loading injection of 70-80 g dissolved in a minimum amount of methylene chloride (200 g) and diluted with heptane (220 g).

F. Galloylation of Benzyl Protected Dimer

The galloylation of 5,7,3′,4′-tetra-O-benzyl-epicatechin-(4β,8)-5,7,3′,4′-tetra-O-benzyl epicatechin is carried out using tri-O-benzyloxy galloyl chloride in the presence of 4-dimethylamiomethylpyridine in dry pyridine. The crude product is obtained in high yield. The crude product is then purified using a silica gel plug. The only improvement made is decreasing the amount of methylene chloride used for the work up from 40 L to 6 L /122 g of dimer. Thus, excess tri-O-benzyl gallic acide can be removed by filtration prior to final purification.

G. Debenzylation of Protected Dimer and Protected Dimer Digallate

1. Protected Dimer

Previously, debenzylation of 5,7,3′,4′-epicatechin-(4β,8)-5,7,3′,4′-catechin was achieved under hydrogenolysis conditions, i.e., 1 bar hydrogen pressure at room temperature using excess 20 wt. % palladium hydroxide on carbon (50% wet, 30-40 wt.%) as a catalyst and using a mixture of tetrahydronfuran:methanol: water (2:2:0.1, v/v/v) as the solvent. The product was purified by reverse phase preparative HPLC and isolated in low yields (<50%).

Palladium black is less advantageous than palladium hydroxide on carbon.

Changing the solvent mixture used in the above room temperature hydrogenolysis to ethyl acetate:methanol:water (1:1:2, v/v/v) permits isolation of the product from the aqueous layer which contains 88% of the desired product. Hydrogenolysis is successfully performed using 20 wt.% palladium hydroxide (30 wt. %, 50% wet) in a biphasic solvent system of water:ethyl acetate (1:1 to 1:3, v/v) at 15 psi hydrogen pressure for 4 hours. After removing the catalyst by filtration, the aqueous layer is separated from the organic layer, and lyophilized. The desired product is isolated in quantitive yield as a fluffy, off-white solid which has a purity of >98%.

Modifying the solvent mixture shows that both ethyl acetate and water are required for the preparation and isolation of the desired product. When water is eliminated from the reaction mixture and only ethyl acetate is used, the yield is low as is the product's purity. When ethyl acetate is eliminated from the reaction mixture and only water is used, no product is formed. Modifying the solvent ratio of ethyl acetate:water 1:3, v/v also permits isolation of the desired product in quantitive yields and at purities >95%, e.g., 97-99%.

2. Protected Dimer Digallate

The debenzylation is carried out as described above. The work up is slightly different with hexane being added to the reaction mixture to aid in the separation of the aqueous and organic layers and back-extractions of the organic layers are performed using warm water (25°-30° C.).

In the examples which follow all parts are by weight unless indicated otherwise, eq. is equivalent, M is mole(s), v is volume, RT is room temperature, h is hours, min is minute(s), HPLC is high pressure liquid chromatography, and TLC is thin layer chromatography. The HPLC results are reported as (% AUC), i.e., percent area under the curve at a wavelength of 280 nm. A standard HPLC system with photodiode array detection and data system is used. A novel analytical column (Phenomenex synergi-4 micron Fuscon-RP 80 angstrom, 150×4.6 mm) is used with the column temperature controlled at 25° C. The mobile phase consists of acetonitride, water, and 0.01% trifluoroacetic acid. Different gradient systems are used for benylated and non-benzylated compounds.

H. Purification of The Protected (4β,8) Epicatechin Dimer

Separation of the 5,7,3′,4′-tetra-O-benzyl-epicatechin-(4β,8)-5,7,3′,4′-tetra-O-benzyl-catechin dimer from the 5,7,3′,4′-tetra-O-benzyl-catechin monomer and the 5,7,3′,4′-tetra-O-benzyl-epicatechin-(4β,8)-5,7,3′,4′-tetra-O-benzyl-epicatechin-(4β,8)-5,7,3′,4′-tetra-O-benzyl-catechin trimer is carried out on a single silica gel column under high pressure, 100-150 psi, 19.6 Kg silica gel on a 30-cm column, 600-650 mL/min flow rate ethyl acetate/heptane (1/3, v/v), monitoring at 280 nm. A total of 70 g of the crude product is dissolved in a minimum amount of methylene chloride (200 g) and diluted with heptane (220 g) before loading on the column.

### EXAMPLES

### Example 1

### Process for Preparing And Purifying of 5,7,3′,4′-Tetra-O-benzyl-(+)-catechin

A dry 12 L, three-necked round bottom flask equipped with a mechanical stirrer, a dropping funnel, a nitrogen inlet, and an internal temperature probe was charged with (+)-catechin (400 g, 1.38 moles, 1.0 eq.) and dimethyl formamide (4 L, 1 g/10 mL, 10 eq.). To this was slowly added potassium carbonate (1430.5 g, 10.38 M, 7.5 eq.) with stirring. The suspension was stirred at RT for ˜30 min. To this was slowly added benzyl bromide (1180.2 g, 6.9 M, 5 eq.) via the addition funnel. A mild exotherm occurred as the internal temperature rose to 30.6° C. from 21.60° C. It took about 4.5 hours to complete the addition of the benzyl bromide. The suspension was stirred at room temperature for ˜18 to 19 h. The consumption of the starting material was monitored by TLC (30% ethyl acetate:heptane, v/v). The reaction mixture was suction filtered through a pad of celite (500-g) to remove the potassium carbonate. The celite pad was washed four times with ethyl acetate (1 L and three times with ethyl acetate (500 mL each). The combined filtrates were sequentially washed two times with 10% aqueous hydrochloric acid (1.5 L), two times with water (1 L), and one time with 30% aqueous sodium chloride (2 L). The organic layer was dried over anhydrous magnesium sulfate (300 g) and filtered. The solvent was removed under vacuum to afford an off-white to light yellow colored semi-solid. The semi-solid was chased or co-evaporated twice with heptane (500 mL each). The crude product was taken up in trichloroethylene (2 L, 1 g/5 mL based on the (+)-catechin starting material, and heated at reflux until a clear orange to red solution was obtained. The solution was allowed to cool to room temperature with agitation and then it was further cooled to −20 to −26° C. in the freezer for ˜56 hours. The solids obtained were suction filtered, washed two times with cold trichloroethylene (−20° C., 500 mL) and once with cold heptane (−20° C., 500 mL). The solids were dried under high vacuum at 50-55° C. for ˜18 hours to produce an off-white to white solid.

The yield was 412 g. (46%). The HPLC purity was 97.76% (AUC).

### Example 2

### Process for Preparing And Purifying (2R)-5,7,3′,4′-Tetrakis(benzyloxy)flavan-3-one

To a solution of 5,7,3′,4′-tetra-O-benzyl-(+)-catechin from Example 1 (440 g, 0.675 mole, 1 eq.) in dichloromethane (3.2 L) was added at once with stirring at room temperature Dess-Martin Periodinane (DMP) reagent (315.4 g, 0.74 mole, 1.1 eq.). Methylene chloride saturated with water (242 mL) was added dropwise over 90 min. The internal temperature gradually increased from 16.0° to 24° C., reaching the maximum during the addition of the dichloromethane (saturated with water) in approximately 50 minutes. The internal temperature then gradually decreased to 20° C. HPLC analysis of the reaction mixture showed complete consumption of the starting material. Subsequently, a saturated sodium bicarbonate (4 L) solution was added slowly, followed by a 10% aqueous solution of sodium thiosulfate pentahydrate (161.5 g/1.6 L in water). The white precipitate formed upon quenching was suction filtered. The filtrate was transferred to a separatory funnel. The organic layer was separated and the aqueous phase was extracted with methylene chloride (650 mL). The combined oganic phases were dried over magnesium sulfate. During the drying process, the reduced DMP reagent precipitated. Once precipitation was complete, the reaction mixture was suction filtered and the solvent was removed under vacuum. The residue was triturated with methanol (600 mL) for ˜1 h at RT, filtered, and the filtrate was redissolved in boiling methylene chloride (800 mL) and diluted with methanol (1.5 L). The amount of the precipitate increased when the reaction mixture reached room temperature. After stirring for ˜18 h at RT and then for ˜1 h at ice bath temperature, the solids were suction filtered and washed four times with methanol (100 mL). The resulting pinkish precipitate was dried in vacuum.

The yield was 336 g (76.6%). The HPLC purity was 94% (AUC).

### Example 3

### Process for Preparing of 5,7,3′,4′-Tetra-O-Benzyl-(−)-Epicatechin under Meerwein-Ponndorf-Verley Reduction Conditions

A 22-L, three-necked round bottom flask equipped with a heating mantle, an overhead stirrer, a thermometer, and a distillation unit was charged with (2R)-5,7,3′,4′-tetrakis(benzyloxy)flavan-3-one from Example 2 (1263 g, 1.95 M, 1 eq.), toluene (10 L), aluminum isopropoxide (796.6 g, 3.9 M, 2 eq.), and isopropanol (5 L) with agitation. After stirring at room temperature for ˜30 minutes, a slightly yellow turbid solution was obtained. A mild endotherm occurred as the internal temperature dropped to 14.3° C. The suspension was heated with continuous stirring. When the internal temperature increased, the suspension became a clear yellow solution. As the internal temperature reached ≧82° C. (which took about 1.75 to 2 h), the distillation of the acetone and isopropanol solvent began. The solvent was collected in a 2 L round bottom flask. After collecting about 400 mL of distillate was collected, a sample was analyzed by HPLC. The results indicated the presence of unreacted starting material. Additional isopropanol was added to the reaction mixture and the distillation was continued. An additional 2200 mL of distillate was collected. Another sample was analyzed. The HPLC results indicated that the starting material was consumed. The reaction mixture was cooled to RT; the internal temperature was 18.8° C. To the reaction mixture was added slowly with stirring 10% aqueous sulfuric acid (v/v, 3 L). The internal temperature rose to 47.2° C. The initial addition of aqueous sulfuric acid resulted in a gel, which dissolved as more acid was added to the reaction mixture with good agitation. At the end of the addition, a clear biphasic solution was obtained. The mixture was allowed to cool to RT and once the internal temperature reached ≦25° C., the reaction mixture was transferred to a separatory funnel and the organic layer was separated. The organic layer was back washed once with 10% aqueous sulfuric acid (v/v, 3 L). The combined aqueous layers were washed once with toluene (3 L). The organic layers were combined, washed once with 20% aqueous sodium chloride (w/v, 2.5 L), dried over sodium sulfate (750 g), and filtered. The solvent was removed under vacuum keeping the bath temperature at ≦45° C. to produce a light yellow semi-solid. The semi-solid was triturated with methanol (˜1 g/8 mL, 8 L) at room temperature for ˜19 h. The solids were suction filtered, washed five times with methanol (500 mL), and dried under high vacuum at 40-45° C. for 20 h.

The yield was 1018.18 g (80.3%). The HPLC purity was 98%.

Upon triturating with methanol (˜1 g/8 mL, 8 L) at room temperature, the diastereomeric selectivity increased from 26:1 to 92:1 for the 5,7,3′,4′-tetra-O-benzyl-(−)-epicatechin:5,7,3′,4′-tetra-O-benzyl-(+)-catechin as determined by HPLC analysis. Crystallization directly from the concentrated reaction mixture followed by trituration with methanol (˜1 g/8 mL) at 50° C. increased the diasteriomeric selectivity to 650:1 as determined by HPLC analysis.

### Example 4

### Preparation of 4-(2-Hydroxyethoxy)-5,7,3′,4′-tetra-O-benzyl-(−)-epicatechin

Under nitrogen and to a stirred solution of 5,7,3′,4′-tetra-O-benzyl-(+)-catechin (30 g, 46 mol, 1.0 eq.) in methylene chloride (300 ml) at room temperature was added anhydrous ethylene glycol (15.4 mL, 276 mol, 6 eq.) and 2,3-dichloro-5,6-dicyano-1,4-benzoquinone (20.9 g, 92 M, 2.0 eq.). The color of the reaction mixture turned green, then almost black. After stirring the reaction mixture at RT for ˜2 hours, a solution of 4-dimethylaminopyridine (11.86 g, 9.7 M, 2.1 eq.) in methylene chloride (70 mL) was added. The reaction mixture was further stirred for an additional 10 min. Silica gel (200 g) was added and the reaction mixture was dried in vacuum for ˜20 h at RT. The silica gel slurry was placed on top of a 400 g silica gel column and the product, was eluted with heptane:ethyl acetate (2:1, v/v,˜14 L). Fractions containing pure product (89.5% AUC), as judged by TLC and HPLC analysis, were combined. The solvent was removed under vacuum. The residue was dissolved in boiling ethyl acetate (200 mL) and upon cooling diluted with heptane (200 mL). Crystallization began in a few minutes. The resulting suspension was vigorously stirred overnight at RT for ˜18 h. The solid was filtered, washed with heptane, and dried in vacuum to afford an off-white solid.

The yield was 15.7 g (48%). The HPLC purity was 98.%.

### Example 5

### Preparing and Purifying of 5,7,3′,4′-Tetra-O-Benzyl-Epicatechin-(4β,8)-(5,7,3′,4′-Tetra-O-Benzyl-Catechin) Dimer

To an ice cold (internal temperature <5° C.) suspension of 5,7,3′,4′-tetra-O-benzyl-(+)-catechin from Example 3 (1189.7 g, 1.83 M, 4.46 eq.) and Bentonite clay K-10 (574 g) in methylene chloride (10 L) under a nitrogen atmosphere was added a solution of the 5,7,3′,4′-tetra-O-benzyl-4-(2-hydroxyethoxy)-(−)epicatechin of Example 4 (290 g, 0.41 mole, 1.0 eq.) in methylene chloride (1000 mL) slowly with stirring at a rate such that the internal temperature was maintained at <6° C. throughout the addition which took ˜1.5 hours. The reaction mixture was stirred at this temperature for an additional 1 h. The internal temperature rose to ˜10° C. The clay was suction filtered through a pad of celite. The clay was then washed with dichloromethane (2 L). The filtrates were combined and the solvent was removed under vacuum to produce an off-white solid. The crude weight was 1483 g (constant weight).

The solid analyzed by HPLC was a mixture of the 5,7,3′,4′-tetra-O-benzyl-(+)-catechin (used in excess), 5,7,3′,4′-tetra-O-benzyl-catechin-(4β,8)-5,7,3′,4′-tetra-O-benzyl-epicatechin dimer, and the 5,7,3′,4′-tetra-O-benzyl-(−)-epicatechin-(4β,8)-5,7,3′,4′-tetra-O-benzyl-epicatechin-(4β,8)-5,7,3′,4′-tetra-O-benzyl-(−)-catechin trimer. The solid was purified by a single column purification under high pressure using 19.6 Kg of silica gel, at a flow rate of 600-650 mL/minute, a wavelength of 280 nm, and a loading injection of 70-80 g (dissolved in minimum amount of methylene chloride and diluted with heptane before loading on the column). The elution of the monomer, dimer, and trimer was monitored at 280 nm. Based upon their different absosption and elution times, monomer, dimer, and trimer fractions were collected. The fractions were analyzed by HPLC and equivalent fractions were combined and the solvent was removed in vacuuo to give the desered products.

### Example 6

### Preparing and Purifying Benzyl-Protected Epicatechin-(4β,8)-Benzyl-Protected-Epicatechin Dimer

A suspension of 5,7,3′,4′-tetra-O-benzyl-(−)epicatechin (306 g, 0.47 M, 4.0 eq.), whose preparation was described in Example 3, and Bentonite clay K-10. (165 g) in methylene chloride (3650 ml) was cooled in an ice bath under a nitrogen atmosphere. To this was slowly added a solution of the 5,7,3′,4′-tetra-O-benzyl-4-(2-hydroxyethoxy) epicatechin of Example 4 in (83.6 g, 0.12 mole, 1.0 eq.) in methylene chloride (150 mL) with stirring keeping the internal temperature at <5° C. throughout the addition. It took ˜2.5 hours for the addition. The reaction mixture was stirred for an additional 1 h at ice-bath temperature. The internal temperature rose to ˜10° C. Completion of the reaction was monitored by HPLC and TLC ethyl acetate:methylene chloride:heptane 1:14:14, v/v/v). The reaction mixture was filtered through a pad of celite to remove the clay. The celite was washed twice with ethyl acetate (20 mL). The filtrates were combined and the solvents were removed under vacuum to afford a foamy solid. Most of the unreacted 5,7,3′,4′-tetra-O-benzyl-epicatechin monomer was isolated by treating the foamy solid with boiling ethyl acetate (750 mL) followed by cooling the solution to room temperature with agitation. The precipitate formed was suction filtered, dried under high vacuum, and analyzed by HPLC. The HPLC purity was >99%. The filtrates were combined and the solvent was removed under vacuum to give 220.0 g of solid 5,7,3′,4′-tetra-O-benzyl-epicatechin monomer. Separation of dimer from the reaction mixture containing 5,7,3′,4′-tetra-O-benzyl-epicatechin-(4β,8)-5,7,3′,4′-tetra-O-benzyl-epicatechin-( 4β,8)-5,7,3′,4′-tetra-O-benzyl-epicatechin trimer was performed using a single wavelength of 280 nm and a silica gel loading injection of 70-80 g of sample dissolved in minimum amount of methylene chloride (200 g) and diluted with heptane (220 g) before loading the column at 100-150 psi pressure using 19.6 Kg of silica gel. The flow rate was 600-650 mL/minute.

### Example 7

### Debenzylation of The Benzyl-Protected Epicatechin-(4β,8)-Catechin Dimer

To a 6 L pressure bottle was added as a catalyst 20% palladium hydroxide on carbon (50% wet, 18 g, 60 wt %). To this was added a solution of the tetra-O-benzyl-(−)epicatechin-(4β,8)-5,7,3′,4′-tetra-O-benzyl-(+)-catechin dimer of Example 5 (27.3. g, 0.021 M) in ethyl acetate (HPLC grade, 100 mL) followed by addition of water (HPLC grade, 300 mL). The bottle was sealed and purged three times with nitrogen (10 psi) and then three times with hydrogen at 15 psi . The reactor was pressurized with hydrogen (15 psi) and stirring was started. After stirring for 3 hours at RT the reactor was vented and purged three times with nitrogen. The reaction mixture was filtered through a cartridge (Millipore, Opticap 4″, 0.22 μm) directly into a separatory funnel containing hexane (100 mL). The aqueous layer was separated. The vessel and cartridge were washed twice with water (1 L). Each time the aqueous layer was separated and combined with the other aqueous layers. The aqueous layers were then poured into two trays and put into the lyophilizer. After 5 days, the trays were removed and placed into a nitrogen glove bag. The product was isolated as a pale yellow solid.

The yield of 13.17 g was quantitative. The HPLC purity was 96%.

### Example 8

### Preparation of The Benzyl Protected Dimer Digallate

To a suspension of tri-O-benzyl gallic acid (206.9 g, 470 M, 5 eq) and dimethyl formamide (1.62 mL, catalytic amount) in anhydrous methylene chloride (3.27 L) was slowly added oxalyl chloride (65.33 g, 44.9 mL, 515 M, 5.5 eq) at RT with agitation under nitrogen. The reaction mixture was stirred for 1 h at RT. Additional oxalyl chloride (4 mL) and dimethylformamide (0.5 mL) were added to the reaction mixture. After 1.5 h, the reaction mixture was concentrated under vacuum. The residue was chased twice with toluene (500 mL). To this was then added a solution of the 5,7,3′,4′-tetra-O-benzyl-epicatechin-(4β,8)-5,7,3′,4′-tetra-O-benzyl-epicatechin dimer from Example 6 (122 g, 93.8 mol, 1 eq) in dry pyridine (2.72 L). The reaction mixture was stirred at RT for 90 h. Water (135 mL) was added to the reaction mixture and the stirring was continued for an additional 4 h at RT. The reaction mixture was diluted with methylene chloride (6 L) and 25% aqueous hydrochloric acid (600 mL). The organic layer was separated and the aqueous layer was extracted with methylene chloride (2 L). The combined organic layers were washed with brine (750 mL) and dried over sodium sulfate (Na₂SO₄). Some of the tri-O-benzyl gallic acid precipitated during the storage at RT was filtered off. The solvent was removed under vacuum to afford a semi-solid, which was purified by a silica gel plug (2.5 Kg) using methylene chloride as the eluent. The crude product was further purified on a silica gel column using heptane:trichloromethane:ethyl acetate 14:14:1, v/v/v) to produce the desired product as an off-white foamy solid.

The yield was 161 g. (80%). The purity was 96%.

### Example 9

### Debenzylation of Dimer Digallate

In a 6 L hydrogenation glass apparatus was added a solution of 5,7,3′,4′-tetra-O-benzyl-3-(3,4,5-tri-O-benzyl-galloyl)-epicatechin-(4β,8)-5,7,3′,4′-tetra-O-benzyl-3-(3,4,5-tri-O-benzyl-galloyl)-epicatechin from Example 9 (23 g, 0.466 M, 96% purity) in ethyl acetate (144 mL) to a suspension of 20% palladium hydroxide on carbon (60 wt % wet, 13.8 g) in water (HPLC grade, 431 mL) at room temperature with stirring. The reaction vessel was purged twice with nitrogen followed by hydrogen. The reaction mixture was allowed to stir at RT in the presence of 15 psi hydrogen for 4 h. After purging the reaction mixture with nitrogen, the reaction mixture was filtered through a filter cartridge (Millipore, 0.22 μm). The cartridge was washed with water (300 mL), ethyl acetate (1 L) and water (1 L). The combined washings were transferred to a 6 L separatory funnel and hexane (200 mL) was added. The organic layer turned cloudy. Addition of the hexane helped in the separation of the layers. The aqueous layer was separated. The cartridge was again washed with ethyl acetate (1 L) and warm water (25°-30° C.) (1 L). Again the aqueous layer was separated. The organic layers were combined and washed with warm water (25°-30° C.) (1 L). The aqueous layers were combined, frozen and lypholized for ˜72 h to afford the epicatechin-(4β,8)-epicatechin dimer as an off-white solid.

The yield was 80%. The purity was 98.44%.

While the invention has been described with respect to certain specific embodiments, it will be appreciated that many modifications and changes may be made by those skilled in the art without departing from the invention. It is intended, therefore, for the appended claims to cover all such modifications and changes as may fall within the true spirit and scope of the invention.

1-26. (canceled)

27. An improved process for preparing (−)-epicatechin-(4β,8)-(+)-catechin dimer or (−)-epicatechin-(4β,8)-(−)-epicatechin dimer by hydrogenating 5,7,3′,4′-tetra-O-benzyl-(−)-epicatechin-(4β,8)-5,7,3′,4′-tetra-O-benzyl-(+)-catechin or 5,7,3′,4′-tetra-O-benzyl-(−)-epicatechin (4β,8)-5,7,3′,4′-tetra-O-benzyl-(−)-epicatechin at room temperature in the presence of excess palladium hydroxide on carbon as a catalyst, wherein the improvement comprises carrying out the hydrogenating in a biphasic solvent consisting essentially of 1 part of ethyl acetate and 3 parts of water (v/v); isolating the dimer by separating the aqueous layer from the reaction mixture; washing the aqueous layer with an organic solvent; and lypholizing the washed aqueous layer.

28. The process of claim 27, wherein the catalyst is 30 wt. % loading of palladium hydroxide on carbon (20 wt. %; 50% wet); and wherein the hydrogen pressure is about 15 psi.

29-32. (canceled)

33. An improved process for preparing (−)-epicatechin-(4β,8)-(−)-epicatechin dimer digallate or (−)-epicatechin-(4β,8)-(+)-catechin dimer digallate, which comprises (a) hydrogenating 5,7,3′,4′-tetra-O-benzyl-3-O-(3,4.5-tri-O-benzylgalloyl)-(−)-epicatechin-(4β,8)-[5,7,3′,4′-tetra-O-benzyl-3-O-(3,4,5-tri-O-benzylgalloyl)]-(−)-epicatechin or 5,7,3′,4′-tetra-O-benzyl-3-O-(3,4,5-tri-O-benzylgalloyl)-(−)-epicatechin-(4β,8)-5,7,3′,4′-tetra-O-benzyl-O-(3,4,5-tri-O-benzylgalloyl)-(+)-catechin at room temperature in the presence of palladium hydroxide on carbon, wherein the improvement comprises (a) filtering the reaction mixture after hydrogenating; (b) washing the filter cartridge with water, with ethyl acetate, and again with water; (c) combining the washings; (d) adding hexane; (e) optionally rewashing the cartridge with ethyl acetate and water; (f) separating the aqueous and organic layers; (g) washing the organic layer with water; (h) separating the aqueous layer; (i) combining the aqueous layers; and (j) lyophilizing the combined aqueous layers.

34. The process of claim 33, wherein the water washing is carried out at about 25° to about 30° C. and wherein the lyophilizing is carried out for about 72 hours at room temperature.

35-37. (canceled)

38. An improved process for preparing 5,7,3′,4′-tetra-O-benzyl-(−)-epicatechin from (+)-catechin comprises the steps of:
(a) benzylating the (+)-catechin at room temperature with 5 equivalents of benzyl bromide in the presence of potassium carbonate and sufficient dimethylformamide to solubilize the potassium carbonate;
(b) oxidizing the compound from step (a) with freshly prepared Dess-Martin periodinane in methylene chloride to form (2R)-5,7,3′,4′-tetrakis(benzyloxy)tlavan-3-one;
(c) isolating the compound formed in step (b) by crystallization from a mixture of methanol and methylene chloride;
(d) stereoselectively reducing the compound from step (c) by reaction with about 200 psi hydrogen in the presence of cerium carbonate and ruthenium (II)-(R)-(+)-2,2′-bis(diphenylphosphino)-1,1′-binapthyl in tetrahydrofuran or in the presence of aluminum isopropoxide in toluene and 2-propanol under Meerwein-Pondorf-Verley conditions to form the 5,7,3′,4′-tetra-O-benzyl-(−)-epicatechin.

39. The process of claim 38, wherein in step (a) the benzyl bromide is added over about 18 to about 24 hours and the reaction mixture is stirred at room temperature for about 18 to 24 hours and wherein the crude 5,7,3′,4′-tetra-O-benzyl-(+)-catechin is purified by dissolving the crude product in hot trichloroethylene, allowing the solution to cool to room temperature, cooling the solution to about −20° C. for about 18 hours, and isolating the purified 5,7,3′,4′-tetra-O-benzyl-(+)-catechin.

40. The process of claim 38, wherein in step (c) the (2R)-5,7,3′,4′-tetrakis(benzyloxy)flavan-3-one is dissolved in boiling methylene chloride, the solution is diluted with methanol and stirred at room temperature for about 18 hours and then at 0° C. for about 1 hour, filtered, washed with methanol, and vacuum dried.

41. The process of claim 38, wherein in step (d) the reduction with cesium carbonate and the ruthenium(II)-(R)-(+)-2,2′-bis(diphenylphosphino)-1,1′-binaphthyl is carried out with about 0.1 equivalent of each at about 40° C. for about 63.5 hours or about 75° C. for about 16 hours.

42. The process of claim 38, wherein, after the reduction with the aluminum isopropoxide in step (d), the crude product is purified by trituration with methanol at room temperature, whereby diastereomeric selectivity is increased from about 26:1 to about 92:1.

43. The process of claim 38, wherein, after the reduction with the aluminum isopropoxide in step (d), the crude product is crystallized directly from the concentrated reaction mixture and then triturated with methanol at 50° C., whereby the diasterometic selectivity is increased to about 650:1.

44. An improved process for preparing 5,7,3′,4′-tetra-O-benzyl-(−)-epicatechin-(4β,8)-(5,7,3′,4′-tetra-O-benzyl-(+)-catechin) dimer or 5,7,3′,4′-tetra-O-benzyl-(−)epicatechin-(4β,8)-5,7,3′,4′-tetra-O-benzyl-(−)-epicatechin dimer comprises the steps of (a) coupling 4-(2-hydroxyethoxy)-5,7,3′,4′-tetra-O-benzyl-(−)-epicatechin with at least 4 equivalents of 5,7,3′,4′-tetra-O-benzyl-(+)-catechin or 5,7,3′,4′-tetra-O-benzyl-(−)-epicatechin at 0° C. using Bentonite K-10 clay as a catalyst in dichloromethane under a nitrogen atmosphere; (b) crystallizing out the excess 5,7,3′,4′-tetra-O-benzyl-(+)-catechin or 5,7,3′,4′-tetra-O-benzyl-(−)-epicatechin by adding ethyl acetate; (c) separating the filtrate; and (d) isolating the dimer after silica gel chromatography using a mixture of heptane, ethyl acetate and chloroform as an eluent.

45. The process of claim 44, wherein the silica gel chromatography is carried out at 100 psi.
